(19) United States
(12) Patent Application Publication
(10) Pub. No.: US 2017/0080009 A1
(43) Pub. Date: Mar. 23, 2017

(54) Oligonucleotide Compositions and Methods for Treating Acute Lymphoblastic Leukemia

(71) Applicants: William H. Gmeiner, Yadkinville, NC (US); Timothy S. Pardee, Winston-Salem, NC (US)

(72) Inventors: William H. Gmeiner, Yadkinville, NC (US); Timothy S. Pardee, Winston-Salem, NC (US)

(21) Appl. No.: 15/311,060

(22) PCT Filed: May 14, 2015
(86) PCT No.: PCT/US15/30848

§ 371 Date: Nov. 14, 2016

Related U.S. Application Data
(60) Provisional application No. 61/993,832, filed on May 15, 2014.

(51) Int. Cl. A61K 31/7072

(52) U.S. Cl. CPC A61K 31/7072

(57) ABSTRACT

F10 is an oligonucleotide based on the thymidylate synthase (TS) inhibitory 5-fluorouracil (5-FU) metabolite, 5-fluoro-2′-deoxyuridine-5′-O-monophosphate. The activity of F10 against preclinical ALL models was determined. F10 treatment resulted in robust induction of apoptosis that could not be equaled by 100 fold more 5-FU. F10 was more potent than Ara-C and doxorubicin against a panel of murine and human ALL cells with an average IC₅₀ value of 1.48 nM (range 0.07 to 5.4 nM). F10 was more than 1000 times more potent than 5-FU. In vivo, F10 treatment resulted in a significant increase in survival in 2 separate syngeneic ALL mouse models and 3 separate xenograft models. F10 also protected mice from leukemia-induced weight loss. In ALL cells made resistant to Ara-C F10 remained highly active in vitro and in vivo. Using labeled F10, it was determined that uptake by the ALL cell lines DG75 and SUP-B15 was rapid and had a profound temperature dependence. Both cell lines demonstrated increased uptake compared to normal, murine, lineage-depleted marrow cells. Consistent with this decreased uptake, F10 treatment did not alter the ability of human HSCs to engraft in immunodeficient mice.

The present invention claims priority under 35 USC 365 and 371 to PCT Application No. PCT/US2015/30848 filed May 14, 2015, which in turn claims priority under 35 USC 119(e) to U.S. Provisional Application No. 61/993,832 filed May 15, 2014, the entire contents of all of which, as well as the accompanying documents are incorporated by reference in their entireties.

The present invention was supported by Award Number P30CA012197 from the National Cancer Institute and NIH CA102532. Accordingly, the federal government has rights in the invention.

### BACKGROUND OF THE INVENTION

Acute lymphoblastic leukemia (ALL) is an aggressive hematologic malignancy wherein an abnormal proliferation of lymphoblasts suppresses normal hematopoiesis resulting in progressive marrow failure and death. There are approximately 6,000 cases per year diagnosed in the United States. This disease has a bimodal age distribution with an initial peak in childhood and second that increases in older adults. While outcomes for ALL in children have improved with an approximately 85-90% cure rate with modern chemotherapy regimens, outcomes in adults are much worse with an estimated 30-45% cure rate. This is worse still in patients over the age of 60 where the 5 year survival rate is estimated at ˜10% and has not significantly improved since the 1980's. This is further complicated by the fact that the median age of adults at diagnosis is ≧60. This inferior outcome is attributable to a combination of increased adverse tumor biology and decreased tolerance of therapy. As a result despite a high initial remission rate most adults are destined to relapse. Once adult patients have relapsed outcomes are particularly dismal. In a large study of adult ALL patients in first relapse; the 5-year survival rate was 7%. The authors stated that most adults with recurrent ALL “cannot be rescued with current therapies”. This is likely the result of the fact that virtually all active therapies are used during first line treatment. There is clearly a need for additional active therapies in ALL.

The development of imatinib to target the BCR-ABL kinase in chronic phase CML has changed the natural history of the disease. This success generated much enthusiasm for the approach in the more genetically complex acute lymphoblastic leukemia (ALL). Unfortunately, the durable responses seen in CML are not reproduced in BCR-ABL positive ALL. Indeed, all previous attempts to target a single oncogenic pathway in ALL have resulted in transient responses with frequent relapse. An alternative approach is to use agents that target “final common pathways”, i.e. processes that must be accomplished to produce additional leukemia cells regardless of driving mutations. In this paradigm, agents are not judged by differential expression of a target, but by the degree of differential uptake.

### SUMMARY OF THE INVENTION

One pathway that the inventors of the present invention believed could be exploited was the increased uptake of oligonucleotides by ALL cells. F10 is a poison deoxy-oligonucleotide that is a 10mer of the TS inhibitory 5-FU metabolite, 5-fluoro-2′-deoxyuridine-5′-O-monophosphate (FdUMP[10]) SEQ ID NO: 1. F10 has been shown to have high activity against preclinical acute myeloid leukemia models by targeting both TS and topoisomerase I (TopoI). Activity of these targets is important for any replicating cell regardless of driving mutations. The present inventors have found that Jurkat cells, a human T cell ALL line, exhibited low nanomolar sensitivity to F10 suggesting F10 may also have utility in ALL. In this invention, the activity, toxicity and uptake of F10 in preclinical models of ALL was determined.

### BRIEF DESCRIPTION OF THE FIGURES

FIGS. 1 A and B show figures that demonstrate that F10 exposure induces apoptosis in ALL cells.

FIGS. 2A-F show figures that demonstrate that F10 is active against multiple ALL models in vivo.

FIGS. 3 A and B show figures that demonstrate F10 is a potent inhibitor of TS and ALL cells express TS and TopoI.

FIGS. 4 A-C show figures that demonstrate that F10 has activity against Ara-C resistant ALL in vivo and in vitro.

FIGS. 5 A-C show figures that demonstrate that F10 is well tolerated and does not injure human HSCs.

FIGS. 6 A-C show figures that demonstrate that F10 is rapidly taken up by ALL cells in vitro and in vivo.

FIG. 7 shows a graph showing that F10 is well tolerated by C57Bl/6 mice.

FIGS. 8 A and B show figures that demonstrate that F10 uptake can be competed by unlabeled drug and is highly temperature dependent.

### DETAILED DESCRIPTION OF THE INVENTION

The present invention relates to treating individuals with a high risk of cancer with compositions that comprise oligonucleotides. In one embodiment, the invention relates to treating individuals with an oligonucleotide that comprises F10. In one embodiment, the cancer that can be treated comprises leukemia. In an embodiment, the cancer (or leukemia) comprises acute lymphoblast leukemia (ALL).

### Materials and Methods:

### Cell Culture and Viability Assays

B6 ALL cells were a kind gift of Dr Nidal Boulos at St Jude Children's Research Hospital, Memphis, Tenn. All human cell lines were maintained in RPMI media (Gibco, Carlsbad, Calif.) supplemented with 10% FBS, penicillin and streptomycin. Cells were grown at 37° C. with 5% CO₂. Viability assays were done using the Cell Titer-Glo assay (Promega, Madison, Wis.) according to the manufacturer's protocol or by Trypan blue exclusion assay using the Countess cell counting system (Invitrogen, Carlsbad, Calif.). All murine cells were maintained in stem cell media (40% DMEM, 40% IMDM, 20% FBS, supplemented with murine SCF to 10 ng/ml, murine IL6 to 2 ng/ml, and murine IL3 to 0.4 ng/ml).

### Normal Human HSCs

All samples were collected under an IRB-approved protocol. All patients gave written informed consent. All samples were obtained during clinically-indicated procedures. Cells were obtained from GM-CSF-primed apheresis of peripheral blood, Ficoll separated, and stored at −80 until use. HSCs were obtained from healthy allogeneic stem cell transplant donors.

### Western Blots and Immunofluorescence

Samples were lysed in Laemmli buffer, separated by SDS-PAGE, and transferred to an Immobilon PVDF membrane (Millipore, Billerica, Mass.). Antibodies against p53 (IMX25, 1:1000; Leica Microsystems), TS (#35-5800, 1:1000; Invitrogen), Topoisomerase I (556597, 1:2000; BD Pharmingen), Caspase 3 (9662, 1:2000; Cell Signaling) and actin (AC-15, 1:5000; Abcam) were used. For ECL westerns secondary antibodies were anti-mouse (7076, 1:5000; Cell Signaling) or anti-rabbit (7074, 1:5000; Cell Signaling). For immunofluorescence studies of phosphorylated γH2AX, cells were fixed with 4% NBF, permeabilized with PBS containing 0.2% Triton-X 100, and probed with anti-phosphoH2AX (#2577, 1:100; Cell Signaling Technologies) followed by donkey anti-rabbit Alexa Fluor 594 conjugated antibody (1:500, A-21207; Invitrogen) and visualized via fluorescence microscopy.

### In Vivo Treatment Studies

The Wake Forest University Institutional Animal Care and Use

Committee approved all mouse experiments. Luciferase-tagged leukemia cells were transplanted into 6- to 8-wk-old C57Bl/6 or Balb/c recipient mice by tail-vein injection of 1×10⁶ viable cells. Mice were monitored by bioluminescent imaging on day 6. Imaging was performed using an IVIS100 imaging system (Caliper LifeSciences, Hopkinton, Mass.). Mice were injected with 150 mg/kg D-Luciferin (Gold Biotechnology, St. Louis, Mo.), anesthetized with isoflurane, and imaged for 2 min. For the xenograft studies 6-8-wk old nude mice were subcutaneously injected with 2×10⁶ cells mixed with 200 μl matrigel (BD Bioscience). Chemotherapy was initiated upon detection of clear signals or reproducibly palpable flank tumors. Mice were treated with 300 mg/kg FdUMP[10] by tail vein injection. Control animals were injected with PBS. Repeat luciferase imaging or tumor volume measurements were performed following treatment.

### Toxicology Studies and Murine Bone Marrow Transplantation

Normal Balb/c mice were treated with identical dose, schedule and route of each drug as in the efficacy studies (i.e. Day 1, 3, 5 and 7). 72 hours after the last dose, animals were sacrificed, bilateral femoral cells harvested and organs fixed in 10% neutral buffered formalin followed by routine tissue processing and sectioning for hematoxylin and eosin staining. Slides were then reviewed by a veterinary pathologist using a Nikon Eclipse 50i light microscope. Photographs of tissues were taken using the NIS Elements D3.10 camera and software system. For transplant studies transgenic NSG (Jax labs) mice were irradiated to 2.7 gray and injected with 2.6×10⁶ normal human HSCs. After 60 days recipient femoral bone marrow was harvested and stained with APC-conjugated anti-human CD45 antibody (BD Pharmingen, San Diego, Calif.) and analyzed by flow cytometry.

### Apoptosis Assays

Cells were seeded in 6-well μlates at 25,000 cells/ml in 3 mls, grown for 2 days and treated with the indicated drug. After centrifugation and washing in cold PBS cells were stained with PI (Sigma Aldrich, St. Louis, Mo.) and APC-conjugated Annexin V in a binding buffer (0.1 M Hepes (pH 7.4), 1.4 M NaCl, and 25 mM CaCl2 solution) (BD Pharmingen, San Jose, Calif.) according to the manufacturer's protocol. Flow cytometric analysis was conducted on a Accuri flow cytometer (BD Biosciences).

### TS Catalytic Activity

Cells were plated at 1.5×10⁶ cells in 100 mm2 μlates and grown overnight in RPMI 1640 medium with 20% FBS. Cells were exposed to the indicated drug for 8, 16, 24, or 48 hours. Cells were then harvested in 25 mM Tris-HCl, pH 7.4 with Complete Protease Inhibitor Cocktail (Roche), put through 2 freeze/thaw cycles, and vortexed. The lysates were centrifuged at 10,000×g for 10 minutes at 4° C. TS assays were performed in a final volume of 200 μl containing 75 μM 5,10 methylene tetrahydrofolate in 0.5 M NaOH (Schircks Laboratories, Switzerland), 10 μM dUMP, 200,000 dpm of 3H-dUMP (Moravek Biochemicals), 100 μM 2-mercaptoethanol, and 25 mM KH₂PO₄, pH 7.4. Cell lysate (400 μg of protein) was added to the reaction buffer. Reactions were incubated at 37° C. for 30 minutes and stopped by addition of 100 μl of 20% TCA, incubated for 5 minutes on ice. 200 μL of activated charcoal solution (10 g activated charcoal, 0.25 g BSA, 0.25 g dextran sulfate, in 100 ml of water) was added, vortexed and maintained at room temperature for 10 minutes. Reactions were centrifuged at 10,000×g for 30 minutes. 200 μL aliquots of the supernatant were read by scintillation counting. All reactions were repeated a minimum of three times.

### Statistical Analysis:

All means were compared by two tailed student's T test. Survival curves were estimated by the Kaplan-Meier method and p values were determined by the log rank test. P values below 0.05 were considered significant. Analysis was performed using Graph Pad Prism version 5.02 (Graph Pad Software Inc).

### Results:

In light of the below explained figures, those of ordinary skill in the art will recognize that the present invention relates to various embodiments that can be explained by observing and/or studying the figures as well as minor modifications thereof. Applicants herein describe what is shown in the figures and then below explain how the figures relate to the results and/or various embodiments of the present invention.

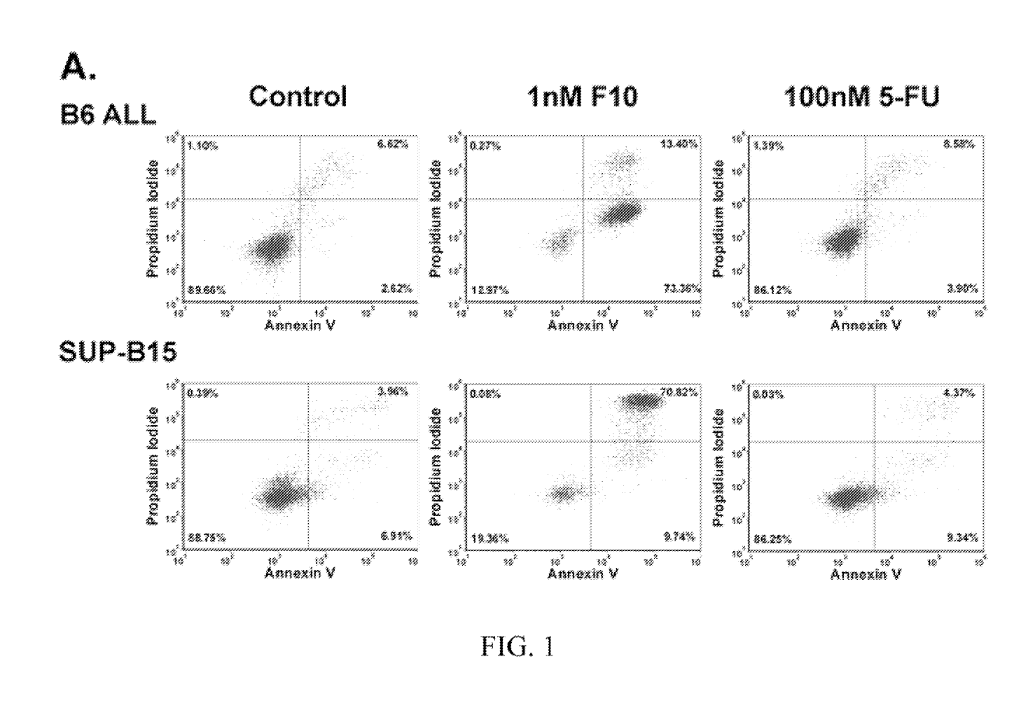

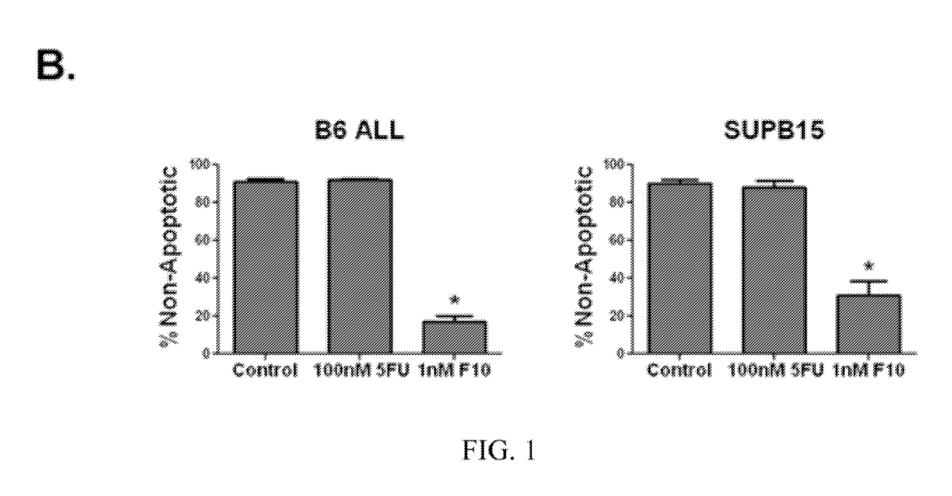

FIGS. 1 A and B show figures that demonstrate that F10 exposure induces apoptosis in ALL cells. FIG. 1 A) Flow cytometry of Annexin V assay. B6 ALL or SUP-B15 ALL cells were exposed to the indicated amounts of F10 or 5-FU for 72 hours and assessed for apoptosis by annexin V and propidium iodide staining. B) Quantitation of non-apoptotic cells. Quantitation of the above from 3 independent experiments. 5-FU treatment was not significantly different from controls. *=p value <0.001.

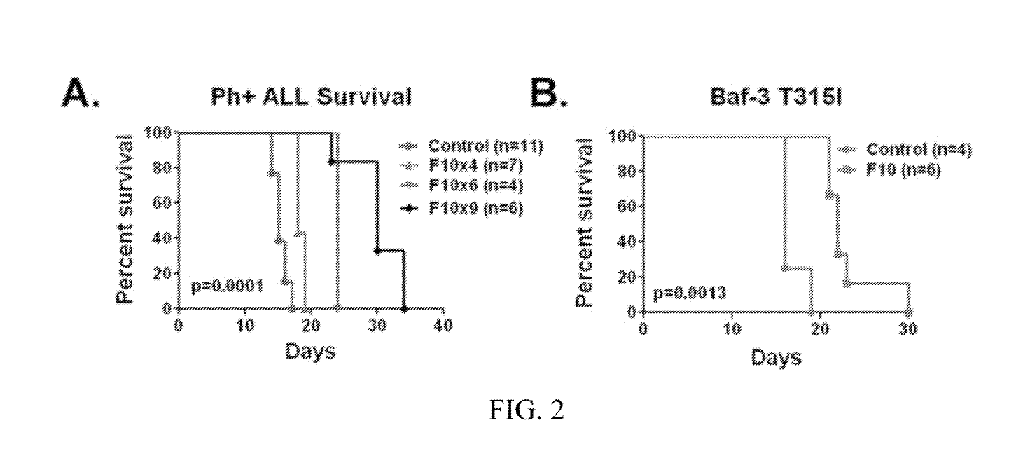

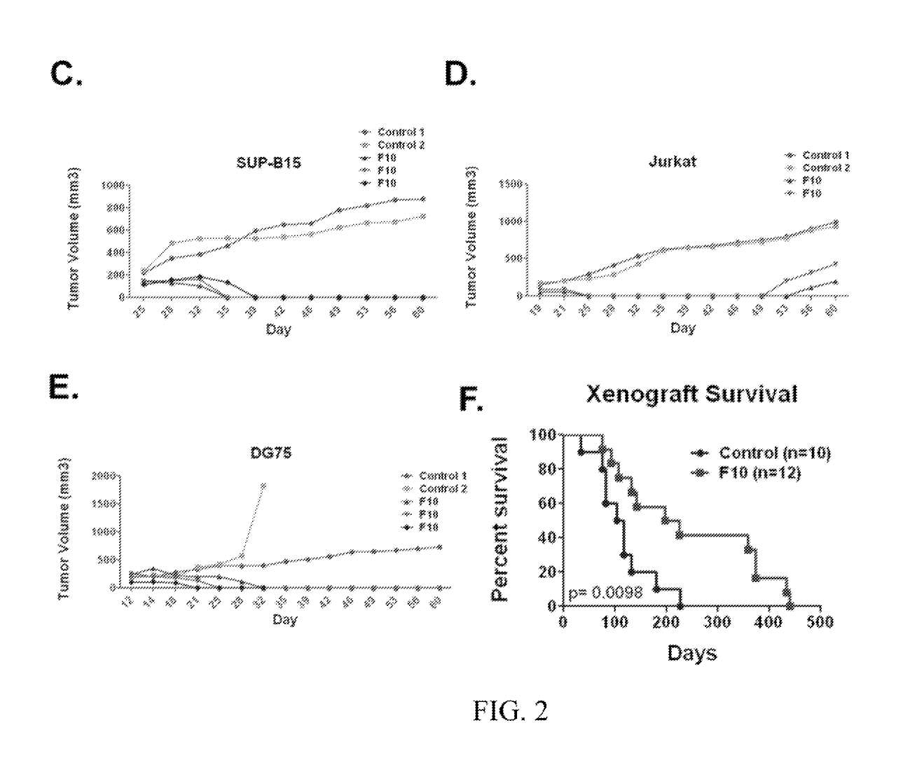

FIGS. 2A-F show figures that demonstrate that F10 is active against multiple ALL models in vivo. A) Survival of C57Bl/6 mice injected with B6 ALL cells and treated with saline or F10 at 300 mg/kg as indicated. B) Survival of Balb/c mice injected with Baf-3 cells expressing the T315I variant of BCR-ABL treated with saline or F10 at 300 mg/kg QOD ×4 doses as indicated. C) Volumes of SUP-B15 tumors in nude mice treated with saline or F10 at 300 mg/kg QOD ×5 doses as indicated. D) Volumes of Jurkat tumors in nude mice treated with saline or F10 at 300 mg/kg QOD ×5 doses as indicated. E) Volumes of DG-75 tumors in nude mice treated with saline or F10 at 300 mg/kg QOD ×5 doses as indicated. F) Survival of all xenograft bearing mice from time of injection treated with saline or F10 at 300 mg/kg QOD ×5 doses as indicated. All p values were derived from log rank tests.

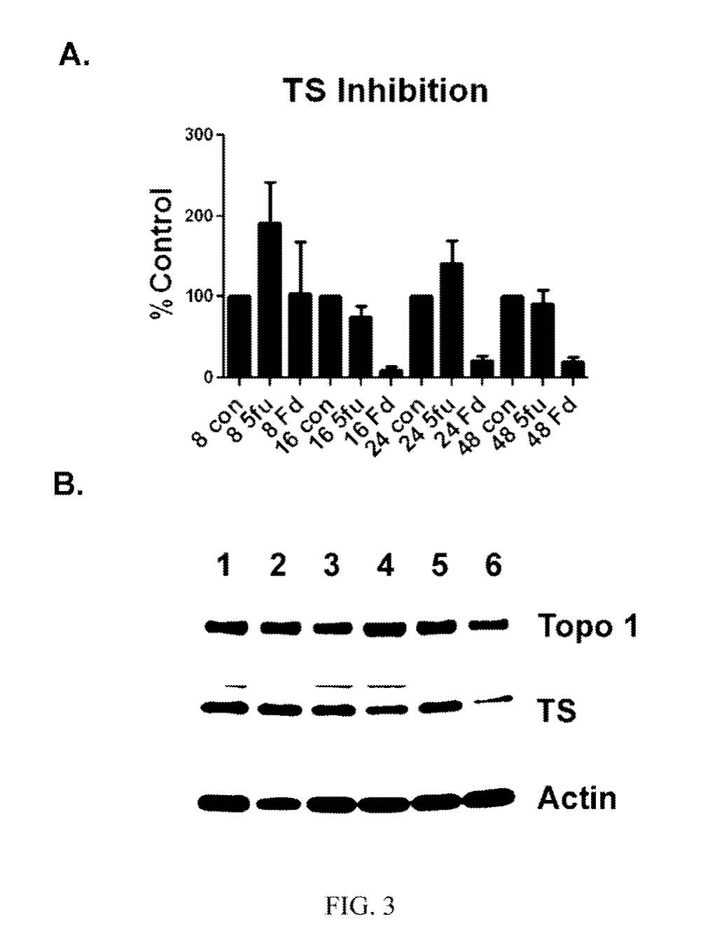

FIGS. 3 A and B show figures that demonstrate F10 is a potent inhibitor of TS and ALL cells express TS and TopoI. (A) TS inhibition assay. Jurkat cells were exposed to 10 nM FdUMP[10] or 100 nM 5-FU for the indicated time, lysed and assayed for TS activity. Activity is plotted as percentage of control. B) Western blot. ALL cells were lysed and extracts blotted for TS, TopoI or actin as indicated. 1=B 6 ALL, 2=CCRF-CEM, 3=DG75, 4=Jurkat, 5=Molt-4, 6=SUP-B15.

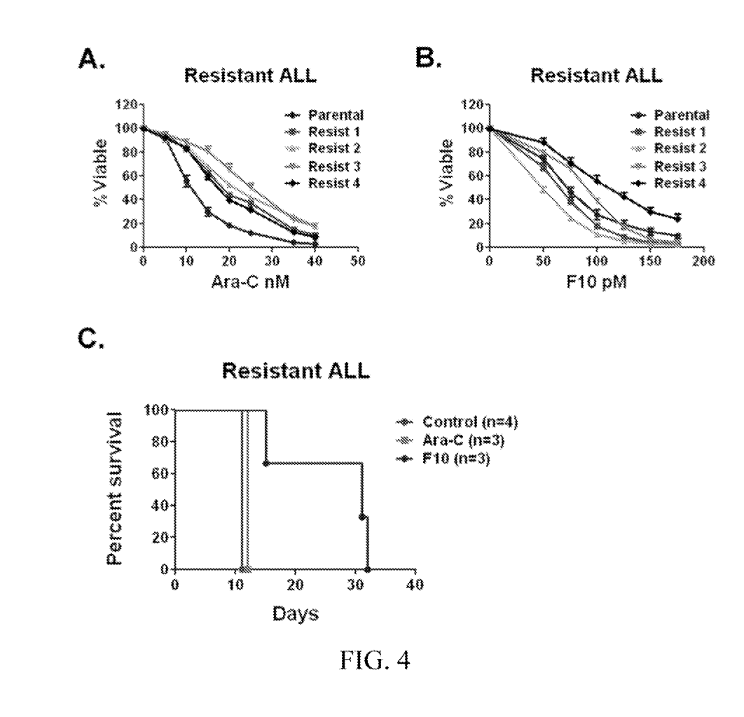

FIGS. 4 A-C show figures that demonstrate that F10 has activity against Ara-C resistant ALL in vivo and in vitro. A) Cell viability assays. Parental B6 ALL cells or Ara-C resistant lines were treated with a titration of Ara-C for 72 hours and viability was assessed. B) Parental B6 ALL cells or Ara-C resistant lines were treated with a titration of F10 for 72 hours and viability was assessed. C) Survival of C57Bl/6 mice injected with Ara-C resistant B6 ALL cells treated with saline, F10 at 300 mg/kg or Ara-C at 100 mg/kg QOD as indicated. p value derived from log rank test.

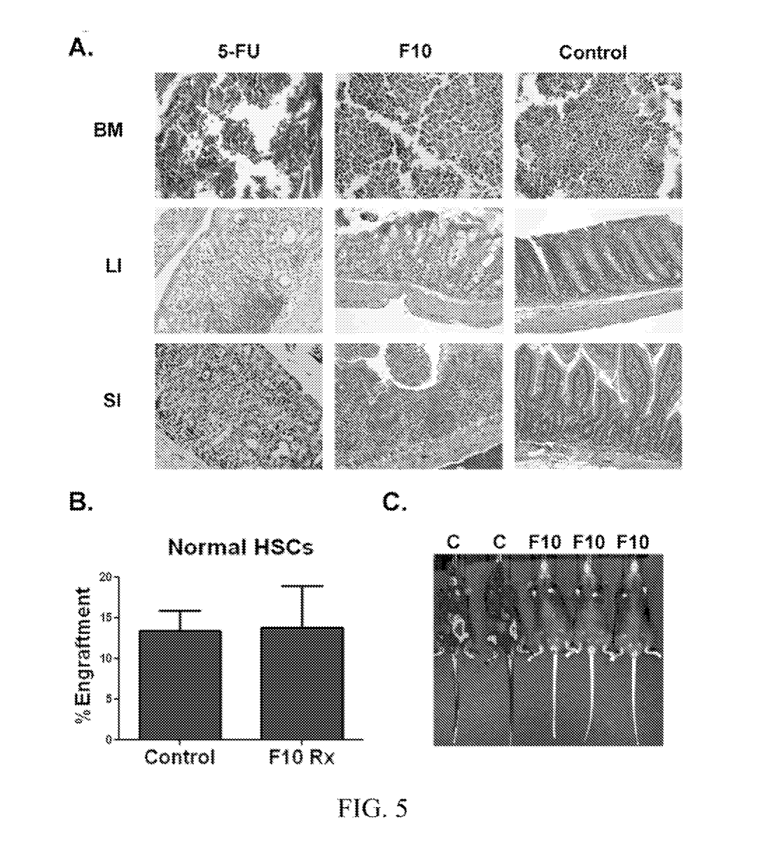

FIGS. 5 A-C show figures that demonstrate that F10 is well tolerated and does not injure human HSCs. A) Hematoxylin and eosin staining of organs from Balb/c mice treated with either saline, F10 at 300 mg/kg or 5-FU at 121 mg/kg QOD ×4 as indicated. Animals were sacrificed 72 hours following their last dose. BM=bone marrow, LI=large intestine, SI=small intestine. B) Engraftment of human HSCs. Human HSCs were treated with saline or 50 nM F10 for 24 hours as indicated and then injected into NSG mice. After 60 days mice were euthanized and bilateral femur cells were assayed for engraftment. C) Bioluminescence of C57Bl/6 mice injected with B6 ALL cells treated with saline (C) or 50 nM F10 (F10) for 24 hours.

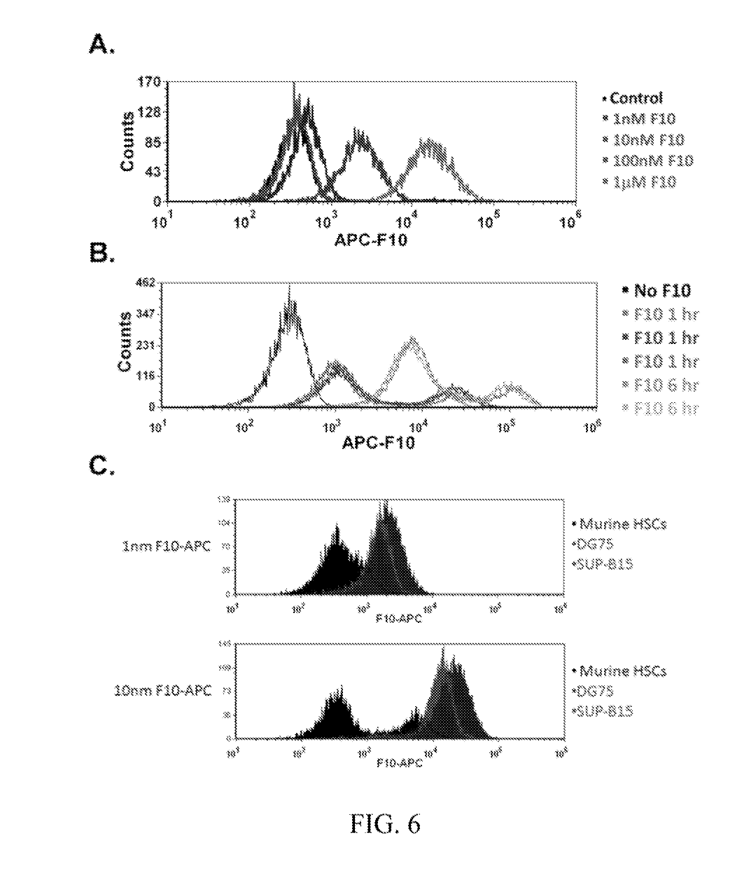

FIGS. 6 A-C show figures that demonstrate that F10 is rapidly taken up by ALL cells in vitro and in vivo. A) SUP-B15 cells were incubated with the indicated amounts of APC labeled F10 for 30 minutes and assayed by flow cytometry. B) In vivo uptake of APC labeled F10. Moribund mice injected with B6 ALL cells were treated with 2 mg/kg APC labeled F10 and bilateral femur cells were harvested after 2 or 6 hours as indicated. C) ALL cells take up F10 more rapidly than normal murine HSCs. DG75, SUP-B15 or lineage depleted femur cells from C57Bl/6 mice were treated with the indicated amount of APC labeled F10 for 2 hours and assayed by flow cytometry.

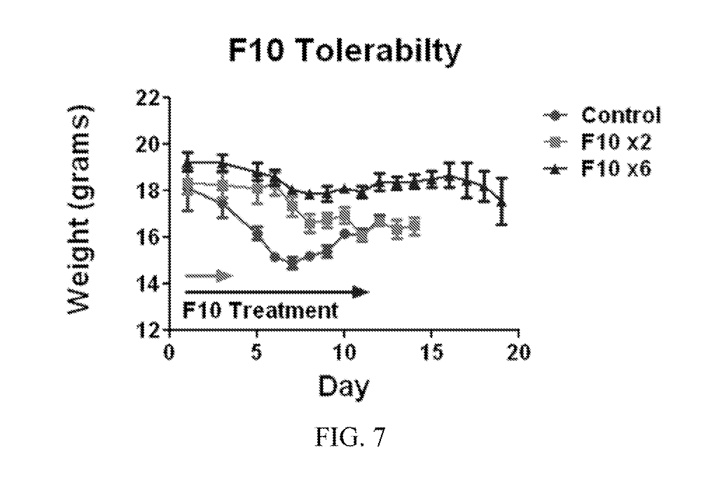

FIG. 7 shows a graph showing that F10 is well tolerated by C57Bl/6 mice. Animals were injected with B6 ALL cells and treated with saline or F10 at 300 mg/kg QOD for 2 or 6 doses as indicated and weights were followed.

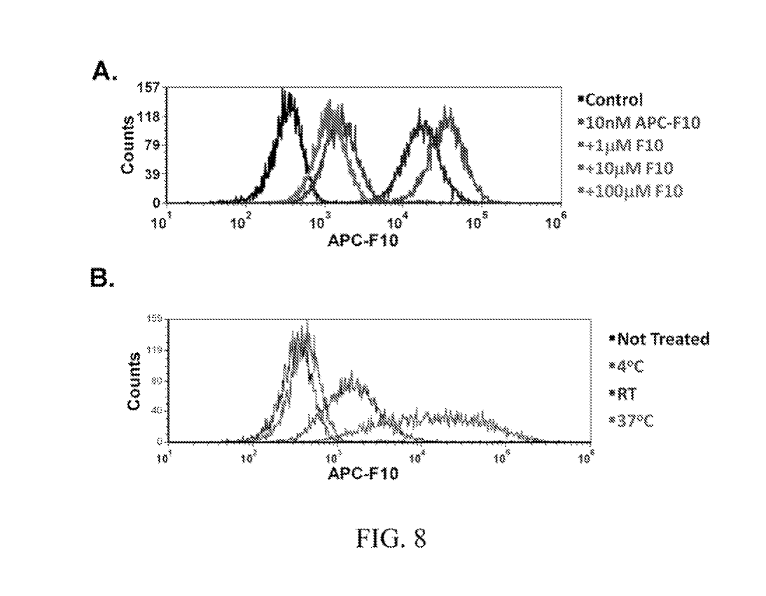

FIGS. 8 A and B show figures that demonstrate that F10 uptake can be competed by unlabeled drug and is highly temperature dependent. A) SUP-B15 cells were incubated with 10 nM APC labeled F10 and the indicated amount of unlabeled drug for 2 hours and then assayed by flow cytometry. B) SUB-B15 cells were treated with 10 nM APC labeled F10 and incubated at the indicated temperature for 4 hours. Cells were then assayed by flow cytometry.

### F10 is Highly Active Against ALL In Vitro.

F10 demonstrated high potency cell killing with the human T cell ALL line, Jurkat. In order to assess the activity of F10 a panel of human and mouse ALL cell lines were exposed to a titration of F10, 5-FU, cytarabine or doxorubicin. After 72 hours viability was assessed and the IC₅₀ values for each was determined. The average IC₅₀ value of F10 for all cell lines was 1.48 nM (range 0.07 to 5.4 nM). In all cases F10 was the most potent agent tested and for all cell lines tested was more than 1000 times more potent than 5-FU (Table 1). In AML, F10 exposure results in a robust induction of apoptosis. To see if ALL cells respond in a similar fashion murine and human ALL cells were exposed to F10 and assessed induction of apoptosis. After 72 hour exposure there was a robust induction of Annexin V and PI positive cells when treated with 1 nM F10 that could not be equaled by 100 nM 5-FU despite 10× more fluoropyrimidine content (FIG. 1A+B). This demonstrates that F10 is handled as a distinct biochemical entity and is not just another vehicle to deliver 5-FU.

### Table IC₅₀ Values for F10, 5-FU, Doxorubicin and Cytarabine

TABLE 1

| Treatment | B6 ALL | | DG75 | | Molt-4 | | SUP-B15 | | CCRF-CEM | |
|---|---|---|---|---|---|---|---|---|---|---|
| F10 | 0.07 | nM | 4.12 | nM | 1.88 | nM | 0.21 | nM | 1.14 | nM |
| | (0.06 to 0.08) | | (2.79-6.10) | | (1.74-2.04) | | (0.20-0.22) | | (0.82-1.58) | |
| 5-FU | 799 | nM | 13,980 | nM | 4,963 | nM | 425.3 | nM | >50,000 | nM |
| | (632-1,010) | | (7,425-26,310) | | (3,933-6,262) | | (117.7-1537.0) | | | |
| Doxorubicin | 4.84 | nM | 150.3 | nM | 19.48 | nM | 19.40 | nM | 36.29 | nM |

| | (3.98-5.90) | | (131.7-171.5) | | (18.0-21.1) | | (16.6-22.7) | (21.7-60.7) | |
|---|---|---|---|---|---|---|---|---|---|
| Cytarabine | 11.93 | nM | 64.28 | nM | 10.63 | nM | ND | 8.32 | nM |

| | | | |
|---|---|---|---|
| (12.8-15.2) | (41.7-99.0) | (9.7-11.6) | (7.73-8.96) |

95% confidence intervals are shown in the parenthesis.
Cells were subjected to 72 hour exposures prior to viability.
ND = Not Determined

### F10 is Highly Active Against Multiple Preclinical ALL Models In Vivo.

F10 exhibited high potency against ALL cells in vitro however many variables present in patients are not accounted for in these types of assays. In order to be of clinical utility F10 must be able to induce ALL cell death when the cells are in their appropriate bone marrow microenvironment, with transient exposure as patients clear the drug and with intact immune systems. The precise, syngeneic, B cell ALL model incorporates all of these factors. To determine the efficacy of F10 against this model 1×10⁶ B6 ALL cells were injected into 6-8 week old syngeneic C57Bl/6 mice. After confirmation of engraftment by luciferase imaging mice were treated with F10 at 300 mg/kg or saline by tail vein injection every other day (QOD) for 4, 6 or 9 treatments. In as few as 4 treatments there was a highly statistically significant prolongation of survival compared to control treated mice (median OS 15 vs 18 days, p=<0.0001). By 6 treatments median OS increased to 24 days (p=0.0009) and by 9 treatments it double the median OS of controls at 30 days (p=0.0001, FIG. 2A). To make sure the efficacy signal seen was not secondary to some inherent sensitivity in the B6 ALL cell line a second syngeneic model system was tested. In this system Baf-3 cells were infected with an MSCV based virus that expressed the T315 variant of BCR-ABL seen in many cases of relapsed Philadelphia chromosome positive ALL⁸. Infected cells were then injected into Balb/c mice and once engraftment was confirmed by bioluminescent imaging, animals were treated with F10 at 300 mg/kg or saline by tail vein injection QOD×4 doses. As before F10 treatment resulted in a significant prolongation of survival (FIG. 2B, p=0.0013). In order to establish that the high efficacy seen in these models was not limited to murine ALL cells the efficacy of F10 against SUP-B15, Jurkat, and DG75 human ALL cell lines in xenograft models was tested. Nude mice were subcutaneously injected with 2×10⁶ cells in matrigel and once a reproducibly palpable tumor was established were treated with F10 at 300 mg/kg or saline by tail vein injection QOD for 5 doses. In all cases F10 treatment resulted in tumor regression (FIG. 2C, D, E) and in several cases resulted in complete eradication of the tumor. More importantly when mice were followed for survival F10 treatment resulted in a significant survival advantage (p=0.0098, FIG. 2F). These data demonstrate the robust in vivo antileukemic activity of F10 and further show that it is active against ALL with diverse lineages and driving mutations.

### F10 is a Potent Inhibitor of TS and TS and Topoisomerase I are Widely Expressed in Human ALL Cells.

The present inventors have demonstrated that F10 is a more potent inhibitor of TS than 5-FU in human AML cells. In order to determine if it had a similar inhibitory activity in ALL cells, treated Jurkat cells with either 10 nM F10 or 100 nM 5-FU. As was seen in AML, F10 treatment resulted in profound and prolonged TS inhibition that could not be achieved by 5-FU despite the identical amount of fluoropyrimidine being present (FIG. 3A). Additionally, F10 is shown to generate trapped topoisomerase I (topol) cleavage complexes resulting in apoptosis. To determine how widely expressed TS and TopoI are in ALL cells western blots were performed against both proteins in a panel of human and murine ALL cell lines. As expected for proteins required for DNA synthesis both were detectable in all cell lines blotted despite their diverse linages and driving mutations (see FIG. 3B). These data demonstrate that F10 is a potent inhibitor of TS and that TS and TopoI are widely expressed in ALL. Taken together these data suggest that F10 is likely to be efficacious for the majority of ALL patients.

### F10 is Active in Cytarabine Resistant ALL Cells.

Induction and consolidation therapy for ALL uses essentially all active agents. As a result the disease relapses responses rates are low and median survival is less than a year. As F10 has distinct cellular targets, the present invention sought to determine if it would have activity against ALL cells that acquired resistance to cytarabine. Cytarabine was chosen as it is widely used in ALL treatment. In order to generate cytarabine resistant cells C57Bl/6 mice were injected with B6 ALL cells and treated them with cytarabine until loss of response. When the mice were moribund bilateral femur cells from 4 separate animals were harvested and they were placed in culture. The relapsed cells were resistant to cytarabine compared with the parental cell line with an approximate doubling of the IC₅₀ from 11 nM to an average of 23 nM (FIG. 4A). In contrast, the average IC50 value for F10 in the resistant lines was 79.98 μM comparable to the parental line IC50 of 68.95 μM (FIG. 4B). In order to further establish the efficacy of F10 against cytarabine resistant cells C57Bl/6 mice were injected with relapsed cells and after engraftment treated the animals with saline, cytarabine at 100 mg/kg QOD or F10 at 300 mg/kg QOD and followed them for survival. Control animals died in a median of 12 days while the cytarabine treated animals lived only a day longer consistent with little benefit to cytarabine treatment. F10 treated animals lived for a median of 31 days, comparable to the survival achieved with the parental cell line (FIG. 4C). These data demonstrate that F10 is not cross resistant with cytarabine and suggests it will be highly efficacious in relapsed ALL.

F10 is Well Tolerated and does not Harm Human Hematopoietic Stem Cells.

F10 was shown to be well tolerated by C57Bl/6 mice when given four 4 doses. In order to determine that F10 tolerability was not specific to the C57Bl/6 strain Balb/c mice were treated with saline, 5-FU at 121 mg/kg or F10 at 300 mg/kg QOD ×4 and then sacrificed animals 72 hours after the last dose. The 5-FU dose was chosen as it delivers the exact same amount of fluoropyrimidine as the F10 dosed at 300 mg/kg. GI tract and bone marrow slides were analyzed by a veterinary pathologist blinded to treatment. In all animals F10 treatment resulted in only minimal marrow toxicity while 5-FU resulted in a pancytopenic and hemorrhagic marrow (FIG. 5A). A similar picture was seen in the GI tract with 5-FU treatment inducing abundant tissue damage and immune cell infiltration while F10 treatment resulted on only a mild increase in apoptotic cells (FIG. 5A). Previous work has demonstrated that F10 did not significantly alter the ability of murine HSCs to engraft into recipients compared to control treated cells. In order to determine if F10 would inhibit the ability of human HSCs to engraft normal human HSCs were treated with saline or 50 nM F10 for 24 hours and then injected the cells into NSG transgenic mice. After 60 days mice were sacrificed, bilateral femur cells harvested and engraftment assessed by staining for human CD45. Pretreatment with 50 nM F10 did not alter the engraftment of human HSCs (FIG. 5B). In contrast when B6 ALL cells were treated with saline or 50 nM F10 for 24 hours prior to injection into syngeneic C57Bl/6 mice, F10 treatment significantly reduced engraftment (FIG. 5C). Finally, to further establish the tolerability of F10 in leukemic mice the weights of animals treated with saline or F10 at 300 mg/kg were followed for 2 or 6 doses. Treatment of F10 protected the mice from leukemia induced weight loss during and for several days after completion of treatment (FIG. 7). These data demonstrate that F10 has little toxicity towards human HSCs, murine marrow and GI tract while it is highly potent against murine ALL initiating cells.

### F10 is Efficiently Taken Up by ALL Cells In Vitro and In Vivo.

It has been demonstrated that leukemia cells take up oligonucleotides with greater efficacy than normal white blood cells. In order to determine the uptake characteristics of F10 human ALL cells were incubated with a fluorescently labeled analog. When DG75 and SUP-B15 cells were incubated with APC conjugated F10 there was a rapid increase in the mean fluorescence intensity with detectable uptake in as little as 30 minutes (FIG. 6A). Furthermore, this uptake could be competed with unlabeled F10 and had profound temperature dependence suggestive of an active carrier mediated process (FIG. 8A+B). To confirm rapid uptake occurred in vivo highly leukemic C57Bl/6 mice were injected with APC conjugated F10 and harvested bilateral femur cells after 2 and 6 hours. There was detectable uptake after 2 hours that increased at 6 hours (FIG. 6B). In order to assess if the differential toxicity seen between normal marrow cells and ALL cells was related to uptake linage depleted murine marrow cells, DG75 and SUP-B15 cells were incubated with APC labeled F10. Both ALL cell lines had a more rapid uptake of F10 compared to the linage depleted marrow cells (FIG. 6C). These data demonstrate a rapid uptake of F10 by ALL cells as compared to normal hematopoietic cells. This differential uptake may contribute to the high efficacy and low toxicity of F10.

### DISCUSSION

Acute lymphoblastic leukemia is an aggressive cancer of the marrow where an abnormal proliferation of lymphoblasts suppresses normal hematopoiesis resulting in marrow failure and death. Despite major advances in the cure rate of ALL in children, results in adults remain unsatisfactory. The current drug development paradigm in oncology is heavily weighted towards the discovery of drug targets uniquely expressed by the tumor cells. The belief is that specific inhibition of such targets will result in cancer cell death while sparing normal cells. A classic example is the targeting of the BCR-ABL tyrosine kinase in CML and ALL. While highly effective in chronic phase CML, BCR-ABL inhibition is only transiently effective in ALL when used as a single agent with frequent relapse. Indeed this appears to be the general rule in genetically complex malignancies like the acute leukemias where such targeted therapy simply selects for resistant subclones that grow out and clinical response is lost. Here a different approach is shown where the drug targets are absolutely required for cell proliferation regardless of driving mutations. In this paradigm, agents are judged by their degree of selective uptake not the selective expression of the target.

F10 is a poison deoxyoligonucleotide that has remarkable antileukemic activity against multiple preclinical AML models. During those previous studies it was observed that F10 was also highly effective against a human T cell ALL line. Additionally, leukemia cells have been shown to have enhanced uptake of deoxyoligonucleotides. For these reasons the efficacy of F10 against preclinical ALL models was determined. In the present invention several important observations should be noted. First, F10 was highly effective against both murine and human ALL cells with low nanomolar to picomolar IC₅₀ values. It also demonstrated in vivo efficacy against 2 separate syngeneic and 3 xenograft ALL models. Second, the targets of F10, TS and TopoI, are widely expressed in ALL cell lines. F10 displayed potent and prolonged TS inhibition in ALL cells that could not be equaled by 5-FU. Third, F10 had remarkable activity against ALL cells that were resistant to cytarabine both in vitro and in vivo demonstrating a lack of cross resistance between the agents. Fourth, F10 was very well tolerated by Balb/c mice. Importantly, it did not affect the ability of normal human HSCs to engraft in immunocompromised mice while severely impairing the ability of murine ALL cells to engraft. Finally, F10 was rapidly taken up by ALL cells in a highly temperature dependent fashion that could be competed by unlabeled compound suggesting receptor mediated active transport. Further studies to characterize the uptake mechanism of F10 are ongoing.

F10's unique mechanism of action results in incorporation of dUMP and FdUMP into DNA in actively replicating cells. As a result once cells have completed S phase they are can no longer be rescued by exogenous thymidine and undergo apoptotic cell death. This apoptotic response does not appear to be dependent on p53. These characteristics suggest F10 may have activity in hard to treat ALL subsets with loss of p53. Additionally, F10 and its metabolite FdUMP are chemically distinct from other nucleoside analogs used to treat ALL and should not be subjected to dephosphorylation by cytosolic 5′-nucleotidase II (NT5C2). This is of interest as activating mutations in NT5C2 have been found in 20% of relapsed T cell ALL patients.

In addition F10 has favorable physical properties with the same strait forward synthesis, solubility and stability as standard DNA oligos. F10 could be produced in lyophilized vials and stored for extended periods before being reconstituted in saline and injected into patients. If it's low toxicities are confirmed in human studies F10 would make an ideal agent for use in developing countries where the outcomes of leukemia patients are suboptimal.

The use of therapeutic oligonucleotides to target specific genes has met with some success in the treatment of leukemia. F10 has several advantages when compared to this approach as it directly targets TS and TopoI without the need of base pairing to its target or shRNA processing. As opposed to targeting antiapoptotic or driving oncogenes that could result in the outgrowth of resistant sub-clones the function of F10's targets are essential for cell replication regardless of driving mutations.

In recent studies the use of agents that harness the immune system to target ALL cells have shown remarkable activity. Several studies using modified T cells that express anti-CD19 chimeric antigen receptors have shown significant activity in ALL patients. Additionally, the use of a bi-specific antibody, blinatumomab, that brings CD3 positive T cells and CD19 positive ALL cells together also has remarkable activity in relapsed B cell ALL. These results have generated significant enthusiasm for the approach. Despite the remarkable success seen in these studies the ability of these immune modifying approaches to treat patients with high disease burden is unclear. Serious side effects including hypotension, high fevers and the need for ICU care have been seen in patients and may be related to the amount of disease present at the time of treatment. Additionally, T cell ALL cannot be treated by these modalities at present. F10 has the ability to treat bulk disease with low toxicity and is effective in T cell as well as B cell ALL. F10 could be used as an initial induction agent with patients who are MRD positive following treatment being selected for additional immunomodulatory therapy. F10 also has the advantage of ease of manufacture and could be produced at relatively low costs.

In sum, F10 exhibits remarkable activity against human and murine ALL cells in vitro and in vivo by inhibition TS and subsequent induction of apoptosis. F10 rapidly internalized by ALL cells and has low toxicity, favorable chemical properties and strait forward synthesis. It is an ideal candidate for future clinical development, particularly as an agent that can be used in developing countries. These data demonstrate agents that target “final common pathways” but with differential uptake can be safe and effective, even against genetically complex and aggressive leukemias.

In an embodiment, the present invention relates to a composition that can be administered to an individual and can be used to treat cancer. In one variation, the composition comprises an oligonucleotide which in one variation comprises 5-fluoro-2′-deoxyuridine-5′-O-monophosphate. In one variation, the oligonucleotide(s) comprise(s) F10. In one variation, it is contemplated and therefore within the scope of the invention that the oligonucleotide length can be a 3-20 mer, or alternatively, a 6-18 mer. In one variation, it is contemplated that F10 is part of a larger molecule (which means that the length of the oligonucleotide is at least a 10 mer). In one variation, F10 is a part of a larger molecule with additional nucleotides and the additional nucleotides are not 5-fluoro-2′-deoxyuridine-5′-O-monophosphate. In one variation, some of the additional nucleotides may be 5-fluoro-2′-deoxyuridine-5′-O-monophosphate. In another variation, all of the additional nucleotides are 5-fluoro-2′-deoxyuridine-5′-O-monophosphate.

In one variation, F10 may be covalently linked to another molecule that is not a nucleotide. For example, antibodies or phospholipids may be used.

In another variation, the cancer is a leukemia. In an embodiment, the leukemia is acute lymphoblastic leukemia. In a variation, the composition is administered to an individual at a dosage wherein F10 is present at a concentration between about 50 mg/kg and 800 mg/kg, or alternatively, at a dosage wherein F10 is present at a concentration between about 100 mg/kg and 500 mg/kg, or alternatively, at a dosage wherein F10 is present at a concentration between about 150 mg/kg and 450 mg/kg, or alternatively, at a dosage wherein F10 is present at a concentration between about 200 mg/kg and 400 mg/kg, or alternatively, at a dosage wherein F10 is present at a concentration at about 300 mg/kg. In one variation, the composition may also comprise a pharmaceutically acceptable diluent, carrier, or excipient.

“Treat” as used herein refers to any type of treatment that imparts a benefit to a subject or patient, including but not limited to reducing symptoms, eliminating symptoms, delaying the onset of symptoms, slowing the rate of progression of symptoms, etc.

Likewise, a “pharmaceutically effective” dose/amount is an amount/dosage that is sufficient to impart a benefit to a subject or patient, including but not limited to reducing symptoms, eliminating symptoms, delaying the onset of symptoms, slowing the rate of progression of symptoms, etc.

“Pharmaceutically acceptable” as used herein means that the compound or composition is suitable for administration to a subject to achieve the treatments described herein, without unduly deleterious side effects in light of the severity of the disease and necessity of the treatment.

The term “about” when used in conjunction with the dosage means a range of +/−10 mg/kg. However, it is contemplated that other error bars may be used, for example, when a dosage is administered, it is contemplated that the error bar may be +/−20 mg/kg, or alternatively, +/−5 mg/kg, or alternatively, +/−3 mg/kg, or alternatively, +/−1 mg/kg.

Classifying ALL tends to be a bit tricky relative to other cancers because in other types of cancer where a solid tumor forms, doctors agree on a set of stages that describe how big the tumor is and where it has spread. Because leukemia (for example, ALL) usually does not form a solid tumor and is found throughout the body when diagnosed, there tends not to be a formal staging system for ALL. Instead, there tend to be general classifications used to describe ALL:

Thus, the classes are usually identified as being Newly diagnosed and untreated, In remission, refractory, and recurrent. These classes are explained below.

Newly diagnosed and untreated. A patient usually has decreased numbers of normal white blood cells, red blood cell, and platelets. However, some patients may have an increased number of white blood cells. Often there are many abnormal lymphoblasts in the blood. The bone marrow contains abnormal lymphoblasts, and the person usually is experiencing symptoms of ALL (listed in the Symptoms and Signs section).

In remission. A patient has received treatment for ALL. The bone marrow contains less than 5% blasts, and the patient has no symptoms. The numbers of normal white blood cells, red blood cells, and platelets are normal.

Refractory. Refractory leukemia means that the disease has not responded to treatment.

Recurrent. Recurrent leukemia has come back after being in remission.

The classification of ALL may lead a physician to alter treatment depending on the results of initial treatment. For example, if ALL is refractory, the physician may amend treatment to increase dosages or to employ combination therapy (discussed infra).

In some embodiments the subject may be one categorized as in a good prognostic risk category (that is, a subject that has a high probability of cure), in other embodiments the subject may be one who is categorized in an intermediate prognostic risk category, and in still other embodiments the subject may be one who is categorized in a poor (or adverse) prognostic risk category.

In an embodiment, the present invention relates to methods of use such as a method of treating cancer or to the use of a pharmaceutically acceptable dose of a composition for treating cancer. In one variation, the composition as enumerated above can be administered to an individual to treat cancer. In one variation, the methods and uses comprise using a composition that comprises an oligonucleotide, which in one variation comprises 5-fluoro-2′-deoxyuridine-5′-O-monophosphate. In one variation, the oligonucleotide comprises F10. In another variation, the cancer of the method and use is a leukemia. In an embodiment, the leukemia may be acute lymphoblastic leukemia. In a variation, the method may encompass administering a composition to an individual at a dosage wherein F10 is present at a concentration between about 50 mg/kg and 800 mg/kg, or alternatively, at a dosage wherein F10 is present at a concentration between about 100 mg/kg and 500 mg/kg, or alternatively, at a dosage wherein F10 is present at a concentration between about 150 mg/kg and 450 mg/kg, or alternatively, at a dosage wherein F10 is present at a concentration between about 200 mg/kg and 400 mg/kg, or alternatively, at a dosage wherein F10 is present at a concentration at about 300 mg/kg.

In an embodiment of the invention, subtypes of All can be treated. For example, the following subtypes of ALL may be treated:

Acute precursor B-cell (pre B-cell) lymphoblastic leukemia

Acute B-cell lymphoblastic leukemia

Precursor T-cell acute lymphoblastic leukemia

Philadelphia chromosome positive (BCR-ABL fusion) acute lymphoblastic leukemia.

In a variation, the treatments as described in the present invention can be used for treating various additional subtypes such as B-cell type subtypes such as Burkitt leukemia or Burkitt lymphoma. In a variation, the compositions of the present invention can be used to treat subtypes of ALL that have been described by what the cells look like when viewed with a microscope, which are called L1, L2, and L3.

In a variation, the treatments of the present invention may be used against biphenotypic acute leukemia (which, in one embodiment, is a hybrid leukemia that contains ALL and another type of leukemia).

In one embodiment, the pharmaceutically effective amount of the composition is administered at room temperature. By room temperature, it is meant close to 25° C. In one variation, when the word “close” is used, this means a temperature that is within +/−3° C. of the enumerated temperature. In one variation, the pharmaceutically effective amount of the composition is administered at a temperature that is close to a human's body temperature (i.e., 37° C.). It should be understood that a temperature range is contemplated and therefore within the scope of the invention wherein the range is +/−5° C. of the enumerated temperature. Alternatively, the range may be +/−10° C. of the enumerated temperature. In one variation, the composition of the present invention may be administered at a temperature that is close to 4° C. and the temperature is allowed to rise after administration.

In one variation, the subject may be treated with a pharmaceutically acceptable amount of oligonucleotide (such as F10) by intravenous injection every other day (QOD) for 3, 4, 6 or 9 treatments. In a variation, the treatment regime may comprise administering treatment every day for between about 3-20 days. Alternatively, treatment may be administered QOD for between about 3-20 treatments (e.g., up to 40 days). In one variation, any of 4, 6 or 9 treatments may be used and then treatment stop for a plurality of months (for example, 6 months, or alternatively, 12 months) whereupon treatment may be repeated for any of 4, 6 or 9 treatments (usually, QOD).

In one variation, the pharmaceutical composition and methods using the composition may contain pharmaceutically acceptable salts, solvates, and prodrugs thereof, and may contain diluents, excipients, carriers, or other substances necessary to increase the bioavailability or extend the lifetime of the compounds/oligonucleotides of the present invention.

Subjects (individuals) that may be treated by the compounds/oligonucleotides and compositions of the present invention include, but are not limited to, horses, cows, sheep, pigs, mice, dogs, cats, primates such as chimpanzees, gorillas, rhesus monkeys, and, humans. In an embodiment, a subject/individual is a human in need of cancer treatment (e.g., treatment for leukemia or treatment for ALL).

The treatment of the present invention may be tailored to address the age of the individual being treated. Because ALL tends to be more prevalent in human patients that are younger than age 20, accounting for 76% of all leukemia diagnosed before that age, the treatment may be tailored to treat younger patients. ALL is especially common in children younger than 5. After a child grows into adulthood, the general risk of ALL rises again after age 50. About four out of every ten ALL diagnoses will be adults. Thus, in an embodiment, the treatment may be tailored to be administered on a subject that is 20 or younger or alternatively, and/or additionally tailored to be administered to a patient that is 50 or older.

The pharmaceutical compositions containing oligonucleotides of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active oligonucleotides in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycethanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and/or flavoring and/or coloring agents.

The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, sterile water for injection (SWFI), Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

In one variation, the formulations of the present invention suitable for parenteral administration may comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s)/oligonucleotides, or a salt thereof, in a unit dosage form in a sealed container. The compound/oligonucleotides or salts thereof is provided in the form of a lyophilizate which is capable of being reconstituted with a suitable pharmaceutically acceptable carrier to form a liquid composition suitable for injection thereof into a subject. The unit dosage form may in one variation comprise from about 10 mg to about 10 grams of the compound/oligonucleotide or salt thereof. When the compound/oligonucleotide(s) or salt thereof is substantially water-insoluble, a sufficient amount of emulsifying agent which is physiologically acceptable may be employed in sufficient quantity to emulsify the compound or salt in an aqueous carrier. One such useful emulsifying agent that may be used is phosphatidyl choline.

Thus, in another embodiment, the present invention provides a pharmaceutical formulation solution comprising oligonucleotides (such as F10) or a salt thereof.

A solution of the invention may be provided in a sealed container, especially one made of glass, either in a unit dosage form or in a multiple dosage form.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

As noted above, the present invention provides pharmaceutical formulations/compositions comprising the active compounds/oligonucleotides (including the pharmaceutically acceptable salts thereof), in pharmaceutically acceptable carriers for oral, rectal, topical, buccal, parenteral, intramuscular, intradermal, intravenous, and/or transdermal administration.

Any pharmaceutically acceptable salt of oligonucleotides (such as F10) may be used for preparing a solution of the invention. Examples of suitable salts may be, for instance, the salts with mineral inorganic acids such as hydrochloric, hydrobromic, sulfuric, phosphoric, nitric and the like, and the salts with certain organic acids such as acetic, succinic, tartaric, ascorbic, citric, glutamic, benzoic, methanesulfonic, ethanesulfonic and the like. In an embodiment, the oligonucleotides (such as F10) is a hydrochloric acid salt including a mono, di, or trihydrochloride.

Any solvent which is pharmaceutically acceptable and which is able to dissolve the oligonucleotides (such as F10) or a pharmaceutically acceptable salt thereof may be used. The solution of the invention may also contain one or more additional components such as a co-solubilizing agent (which may be the same as a solvent), a tonicity adjustment agent, a stabilizing agent, a preservative, or mixtures thereof. Examples of solvents, co-solubilizing agents, tonicity adjustment agents, stabilizing agents and preservatives which may suitable for a solution formulation are described below.

Suitable solvents and co-solubilizing agents may include, but are not limited to, water; sterile water for injection (SWFI); physiological saline; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethyleneglycol 400, propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols; esters of polyoxyethylenated fatty acids; polysorbates, e.g., Tween™, polyoxyethylene derivatives of polypropyleneglycols, e.g., Pluronics™.

Suitable tonicity adjustment agents may include, but are not limited to, pharmaceutically acceptable inorganic chlorides, e.g. sodium chloride; dextrose; lactose; mannitol; sorbitol and the like.

Preservatives suitable for physiological administration may be, for instance, esters of parahydroxybenzoic acid (e.g., methyl, ethyl, propyl and butyl esters, or mixtures of them), chlorocresol and the like.

Suitable stabilizing agents include, but are not limited to, monosaccharides (e.g., galactose, fructose, and fucose), disaccharides (e.g., lactose), polysaccharides (e.g., dextran), cyclic oligosaccharides (e.g., alpha-, beta-, gamma-cyclodextrin), aliphatic polyols (e.g., mannitol, sorbitol, and thioglycerol), cyclic polyols (e.g. inositol) and organic solvents (e.g., ethyl alcohol and glycerol).

The above mentioned solvents and co-solubilizing agents, tonicity adjustment agents, stabilizing agents and preservatives can be used alone or as a mixture of two or more of them in a solution formulation.

In an embodiment, a pharmaceutical solution formulation may comprise an oligonucleotide (such as F10) or a pharmaceutically acceptable salt thereof, and an agent selected from the group consisting of sodium chloride solution (i.e., physiological saline), dextrose, mannitol, or sorbitol, wherein the agent is in an amount of less than or equal to 5%. The pH of such a formulation may also be adjusted to improve the storage stability using a pharmaceutically acceptable acid or base.

In the solutions of the invention the concentration of F10 or a pharmaceutically acceptable salt thereof may be less than 1000 mg/mL, or less than 500 mg/mL, or less than 400 mg/mL, or less than 300 mg/mL and greater than 0.01 mg/mL. In an embodiment, the concentration that is used is the ideal concentration to be sufficiently cytotoxic to the cancer cells yet limit the toxicity on other cells.

Suitable packaging for the pharmaceutical solution formulations may be all approved containers intended for parenteral use, such as plastic and glass containers, ready-to-use syringes and the like. In an embodiment, the container is a sealed glass container, e.g. a vial or an ampoule. A hermetically sealed glass vial is particularly preferred.

According to an embodiment of the present invention, there is provided, in a sealed glass container, a sterile, injectable solution comprising F10 or a pharmaceutically acceptable salt thereof in a physiologically acceptable solvent, and which has a pH of from 2.5 to 3.5. For solution formulations, various compounds/oligonucleotides of the present invention may be more soluble or stable for longer periods in solutions at a pH lower than 6. Further, acid salts of the compounds/oligonucleotides of the present invention may be more soluble in aqueous solutions than their free base counter parts, but when the acid salts are added to aqueous solutions the pH of the solution may be too low to be suitable for administration. Thus, solution formulations having a pH above pH 4.5 may be combined prior to administration with a diluent solution of pH greater than 7 such that the pH of the combination formulation administered is pH 4.5 or higher. In one embodiment, the diluent solution comprises a pharmaceutically acceptable base such as sodium hydroxide. In another embodiment, the diluent solution is at pH of between 10 and 12. In another embodiment, the pH of the combined formulation administered is greater than 5.0. In another embodiment, the pH of the combined formulation administered is between pH 5.0 and 7.0.

The invention also provides a process for producing a sterile solution with a pH of from 2.5 to 3.5 which process comprises dissolving F10 or a pharmaceutically acceptable salt thereof in a pharmaceutically acceptable solvent. Where a pharmaceutically acceptable acid salt of F10 is used the pH of the solution may be adjusted using a pharmaceutically acceptable base or basic solution adding a physiologically acceptable acid or buffer to adjust the pH within a desired range. The method may further comprise passing the resulting solution through a sterilizing filter.

One or more additional components such as co-solubilizing agents, tonicity adjustment agents, stabilizing agents and preservatives, for instance of the kind previously specified, may be added to the solution prior to passing the solution through the sterilizing filter.

Combination treatments that can be used in conjunction with the oligonucleotides/compounds of the present invention (for example, to treat ALL) may include chemotherapy, chemotherapy with stem cell transplant, radiation therapy and/or immunotherapy.

In a further variation, the present invention contemplates combination therapies in which the oligonucleotides (such as F10) of the present invention can be used in conjunction with other cisplatin compounds. The efficacy of this combination therapy is likely to be enhanced because of the different mechanisms and modes of action that first generation cisplatin compounds exhibit relative to the compounds/oligonucleotides of the present invention. It is also contemplated and therefore within the scope of the invention that other anti-neoplastic agents/compounds can be used in conjunction with the compounds/oligonucleotides of the present invention. The anti-neoplastic agents/compounds that can be used with the compounds/oligonucleotides of the present invention include cytotoxic compounds as well as non-cytotoxic compounds.

Examples include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVAC™ and BEXXAR™ (iodine 131 tositumomab).

Other anti-neoplastic agents/compounds that can be used in conjunction with the compounds/oligonucleotides of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction with the oligonucleotides (such as F10) of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the oligonucleotides (such as F10) of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA,); emaxanib, (Pfizer, USA,); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAb, alpha5beta3 integrin, second generation, (Applied Molecular Evolution, USA and Med Immune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Children's Hospital, USA); ATN 161, (Attenuon, USA); ANGIOSTATIN, (Boston Children's Hospital, USA); 2-methoxyestradiol, (Boston Children's Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Children's Hospital, USA and EntreMed, USA); MAb, KDR, (ImClone Systems, USA); MAb, alpha5 betal, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

It should be understood that the present invention is not to be limited by the above description. Modifications can be made to the above without departing from the spirit and scope of the invention. It is contemplated and therefore within the scope of the present invention that any feature that is described above can be combined with any other feature that is described above. Moreover, it should be understood that the present invention contemplates minor modifications that can be made to the formulations, compositions and methods of the present invention. When ranges are discussed, any number that may not be explicitly disclosed but fits within the range is contemplated as an endpoint for the range. For example, if a range of 3-20 is given, every real integer that fits within that range is contemplated as an endpoint that can be used to establish a subset range (e.g., 4, 5, 6, . . . etc. . . . 0.19). The scope of protection to be afforded is to be determined by the claims which follow and the breadth of interpretation which the law allows.

The following references are incorporated by reference in their entirety.
1. Inaba H, Greaves M, Mullighan C G. Acute lymphoblastic leukaemia. Lancet. 2013; 381(9881):1943-1955. Prepublished on 2013 Mar. 26 as DOI 50140-6736(12)62187-4 [pii] 10.1016/S0140-6736(12)62187-4.
2. Pui C H, Mullighan C G, Evans W E, Relling M V. Pediatric acute lymphoblastic leukemia: where are we going and how do we get there? Blood. 2012; 120(6):1165-1174. DOI 10.1182/blood-2012-05-378943.
3. Brenner H, Pulte D, Gondos A. Improvement in survival in younger patients with acute lymphoblastic leukemia from the 1980s to the early 21st century. Blood. 2009; 113(7):1408-1411. 10.1182/blood-2008-06-164863.
4. Marks D I. Treating the “older” adult with acute lymphoblastic leukemia. Hematology Am Soc Hematol Educ Program. 2010; 2010:13-20. Prepublished on 2011 Jan. 18 as DOI 2010/1/13 [pii] 10.1182/asheducation-2010.1.13.
5. Larson R A. Treatment of older patients with acute lymphoblastic leukemia. Annals of Hematology. 2006; 85:79-81.
6. Fielding A K, Richards S M, Chopra R, et al. Outcome of 609 adults after relapse of acute lymphoblastic leukemia (ALL); an MRC UKALL12/ECOG 2993 study. Blood. 2007; 109(3):944-950. Prepublished on 2006 Oct. 13 as DOI blood-2006-05-018192 [pii] 10.1182/blood-2006-05-018192.
7. Druker B J, Guilhot F, O'Brien S G, et al. Five-year follow-up of patients receiving imatinib for chronic myeloid leukemia. N Engl J Med. 2006; 355(23):2408-2417. Prepublished on 2006 Dec. 8 as DOI 355/23/2408 [pii] 10.1056/NEJMoa062867.
8. Foa R, Vitale A, Vignetti M, et al. Dasatinib as first-line treatment for adult patients with Philadelphia chromosome-positive acute lymphoblastic leukemia. Blood. 2011; 118(25):6521-6528. Prepublished on 2011 Sep. 21 as DOI blood-2011-05-351403 [pii] 10.1182/blood-2011-05-351403.
9. Zhao Q, Song X, Waldschmidt T, Fisher E, Krieg A M. Oligonucleotide uptake in human hematopoietic cells is increased in leukemia and is related to cellular activation. Blood. 1996; 88(5):1788-1795. Prepublished on 1996 Sep. 1 as DOI.
10. Pardee T S, Gomes E, Jennings-Gee J, Caudell D, Gmeiner W H. Unique dual targeting of thymidylate synthase and topoisomerase1 by FdUMP[10] results in high efficacy against AML and low toxicity. Blood. 2012; 119(15):3561-3570. Prepublished on 2012 Mar. 1 as DOI blood-2011-06-362442 [pii] 10.1182/blood-2011-06-362442.
11. Boulos N, Mulder H L, Calabrese C R, et al. Chemotherapeutic agents circumvent emergence of dasatinib-resistant BCR-ABL kinase mutations in a precise mouse model of Philadelphia chromosome-positive acute lymphoblastic leukemia. Blood. 2011; 117(13):3585-3595. Prepublished on 2011 Jan. 26 as DOI blood-2010-08-301267 [pii] 10.1182/blood-2010-08-301267.
12. Jennings-Gee J, Pardee T S, Gmeiner W H. Replication-dependent irreversible topoisomerase 1 poisoning is responsible for FdUMP[10] anti-leukemic activity. Exp Hematol. 2013; 41(2):180-188 e184. Prepublished on 2012 Oct. 23 as DOI S0301-472X(12)00438-9 [pii] 10.1016/j.exphem.2012.10.007.
13. Hof J, Krentz S, van Schewick C, et al. Mutations and deletions of the TP53 gene predict nonresponse to treatment and poor outcome in first relapse of childhood acute lymphoblastic leukemia. J Clin Oncol. 2011; 29(23):3185-3193. Prepublished on 2011 Jul. 13 as DOI JCO.2011.34.8144 [pii] 10.1200/JCO.2011.34.8144.
14. Tzoneva G, Perez-Garcia A, Carpenter Z, et al. Activating mutations in the NT5C2 nucleotidase gene drive chemotherapy resistance in relapsed ALL. Nat Med. 2013; 19(3):368-371. Prepublished on 2013 Feb. 5 as DOI nm.3078 [pii] 10.1038/nm.3078.
15. Jacomo R H, Melo R A M, Souto F R, et al. Clinical features and outcomes of 134 Brazilians with acute promyelocytic leukemia who received ATRA and anthracyclines. Haematologica-the Hematology Journal. 2007; 92(10):1431-1432. DOI 10.3324/haematol.10874.
16. O'Brien S, Moore J O, Boyd T E, et al. 5-year survival in patients with relapsed or refractory chronic lymphocytic leukemia in a randomized, phase III trial of fludarabine plus cyclophosphamide with or without oblimersen. J Clin Oncol. 2009; 27(31):5208-5212. Prepublished on 2009 Sep. 10 as DOI JCO.2009.22.5748 [pii] 10.1200/JCO.2009.22.5748.
17. Moore J, Seiter K, Kolitz J, et al. A Phase II study of Bcl-2 antisense (oblimersen sodium) combined with gemtuzumab ozogamicin in older patients with acute myeloid leukemia in first relapse. Leuk Res. 2006; 30(7):777-783. Prepublished on 2006 May 30 as DOI 50145-2126(05)00423-6 [pii] 10.1016/j leukres.2005.10.025.
18. Marcucci G, Stock W, Dai G, et al. Phase I study of oblimersen sodium, an antisense to Bcl-2, in untreated older patients with acute myeloid leukemia: pharmacokinetics, pharmacodynamics, and clinical activity. J Clin Oncol. 2005; 23(15):3404-3411. Prepublished on 2005 Apr. 13 as DOI JCO.2005.09.118 [pii] 10.1200/JCO.2005.09.118.
19. Kalos M, Levine B L, Porter D L, et al. T cells with chimeric antigen receptors have potent antitumor effects and can establish memory in patients with advanced leukemia. Sci Transl Med. 2011; 3(95):95ra73. Prepublished on 2011 Aug. 13 as DOI 3/95/95ra73 [pii] 10.1126/scitranslmed.3002842.
20. Grupp S A, Kalos M, Barrett D, et al. Chimeric antigen receptor-modified T cells for acute lymphoid leukemia. N Engl J Med. 2013; 368(16):1509-1518. Prepublished on 2013 Mar. 27 as DOI 10.1056/NEJMoa1215134.
21. Topp M S, Gokbuget N, Zugmaier G, et al. Long-term follow-up of hematologic relapse-free survival in a phase 2 study of blinatumomab in patients with MRD in B-lineage ALL. Blood. 2012; 120(26):5185-5187. Prepublished on 2012 Oct. 2 as DOI blood-2012-07-441030 [pii] 10.1182/blood-2012-07-441030.
22. Topp M S, Kufer P, Gokbuget N, et al. Targeted therapy with the T-cell-engaging antibody blinatumomab of chemotherapy-refractory minimal residual disease in B-lineage acute lymphoblastic leukemia patients results in high response rate and prolonged leukemia-free survival. J Clin Oncol. 2011; 29(18):2493-2498. Prepublished on 2011 May 18 as DOI JCO.2010.32.7270 [pii] 10.1200/JCO.2010.32.7270.

We claim:

1. A method of treating cancer in an individual in need thereof, wherein the method comprises administering to said individual a pharmaceutically effective amount of a composition comprising F10.

2. The method of claim 1, wherein the cancer is leukemia.

3. The method of claim 2, wherein the leukemia is acute lymphoblastic leukemia.

4. The method of claim 3, wherein the acute lymphoblastic leukemia is selected from the group consisting of acute precursor B-cell (pre B-cell) lymphoblastic leukemia, acute B-cell lymphoblastic leukemia, precursor T-cell acute lymphoblastic leukemia, and Philadelphia chromosome positive (BCR-ABL fusion) acute lymphoblastic leukemia.

5. The method of claim 3, wherein the composition that comprises F10 contains additional nucleotides covalently linked to F10.

6. The method of claim 5, wherein the individual is treated with F10 at a dosage between about 200 mg/kg and 400 mg/kg.

7. The method of claim 6, wherein the individual is treated F10 at a dosage of about 300 mg/kg.

8. The method of claim 3, wherein the pharmaceutically effective amount of the composition is administered at room temperature.

9. The method of claim 3, wherein the pharmaceutically effective amount of the composition is administered at a temperature that is close to a human's body temperature.

10. The method of claim 3, wherein the pharmaceutically effective amount of the composition is administered parenterally.

11. The method of claim 7, wherein the individual is treated one or more times by a treatment set that comprises 3 treatments every other day (QOD).

12. The method of claim 11, wherein the treatment set comprises between 4 and 20 treatments QOD.

13. The method of claim 12, wherein the treatment set comprises 9 treatments QOD.

14. The method of claim 12, where in the individual is treated by a plurality of treatment sets.

15. The method of claim 1, wherein the composition further comprises one or more of a pharmaceutically acceptable diluent, carrier, or excipient.

16. A pharmaceutical composition for treating acute lymphoblastic leukemia in an individual in need thereof comprising F10, and optionally one or more of a pharmaceutically acceptable diluent, carrier, or excipient.

17. The pharmaceutical composition of claim 16, wherein the composition is administered at a dosage that is about 300 mg/kg.

18. The pharmaceutical composition of claim 17, wherein the composition is administered at a temperature that is close to room temperature.

19. The pharmaceutical composition of claim 18, wherein the composition is administered parenterally.

20. The pharmaceutical composition of claim 16, wherein the composition further comprises co-solubilizing agents, tonicity adjustment agents, stabilizing agents or preservatives.
